(19) United States
(12) Patent Application Publication
(10) Pub. No.: US 2025/0017601 A1
(43) Pub. Date: Jan. 16, 2025

(54) METHODS AND DEVICES FOR GUIDED MODALITY CATHETER PLACEMENT

(71) Applicant: Ray King, Houston, TX (US)

(72) Inventor: Ray King, Houston, TX (US)

(21) Appl. No.: 18/711,278

(22) PCT Filed: Nov. 17, 2022
(86) PCT No.: PCT/US2022/080051

§ 371 Date: May 17, 2024

Related U.S. Application Data
(60) Provisional application No. 63/280,588, filed on Nov. 17, 2021.

(51) Int. Cl. A61B 17/17; A61B 17/00; A61B 17/16; A61B 34/20; A61B 90/11

(52) U.S. Cl. CPC A61B 17/1703; A61B 17/1622; A61B 17/1739; A61B 34/20; A61B 90/11; A61B 2017/00292; A61B 2034/2063

(57) ABSTRACT

Catheter placement in the brain has traditionally been done largely blindly, utilizing surface landmarks (such as entry at the mid-pupillary line anterior to the coronal suture, with catheter target aiming at the medial canthus and tragus for the ventriculostomy procedure). This invention relates to methods and devices for placement of catheter(s), more particularly to guided modality for catheter placement, and more particularly to guided modality placement of intracranial catheters using intra-luminal ultrasound, employing a drill, surgical navigation, non-real-time-navigated external guide, or any combination thereof.

### CROSS-REFERENCE TO RELATED APPLICATIONS

This application claims the benefit and priority of U.S. provisional patent application Ser. No. 63/280,588, filed Nov. 17, 2021, the contents of which is hereby incorporated by reference in its entirety.

### FIELD OF THE INVENTION

This invention relates to methods and devices for placement of catheter(s), more particularly to guided modality for catheter placement to reach intended target structure(s) while avoiding traversing unintended structure(s), and more particularly to guided modality placement of intracranial catheters with intra-luminal ultrasound, possibly employing a drill, surgical navigation, external guide, or any combination thereof.

### BACKGROUND OF THE INVENTION

Catheter placement in the brain has traditionally been done largely blindly, utilizing surface landmarks (such as entry at the mid-pupillary line anterior to the coronal suture, with catheter target aiming at the ipsilateral medial canthus and tragus for the ventriculostomy procedure).

Though navigation has added a measure of safety by allowing pre-planning based upon the patient's MRI or CT scan, with avoidance of blood vessels via a planned path, it is susceptible to brain shift and hence resultant inaccuracy; moreover, navigation equipment is both expensive and bulky, as such relegating it to operating rooms vs. the intensive care unit (ICU) where bedside ventriculostomy procedures utilize catheters to cannulate a ventricle. Hence it is rarely used, and the procedure is done blind except for surface landmarks, with a known incidence of bleeds due to hitting a vessel during the path to the ventricle via the brain, or not being able to cannulate the ventricle despite numerous “passes” or attempts, increasing risk of bleed or direct neurological injury or both.

While navigation is an option that may be considered as complementing the current invention, its cost, bulkiness, unreliability with brain shift, and limited availability typically allow other modalities to be considered, such as ultrasound in combination with other medical devices in this procedure.

### SUMMARY OF THE INVENTION

This invention relates to methods and devices for placement of catheter(s), more particularly to guided modality for catheter placement, and more particularly to guided modality placement of intracranial catheters with intra-luminal ultrasound, employing a drill, surgical navigation, or any combination thereof. In general, a device that may drill (with or without use of an external cranial inexpensive and portable guide, such as the Thomale Guide) may benefit from being cannulated, allowing placement of a thin or small imaging or sensing probe, such as, for example, a thin ultrasound probe, via a cannulated portion (or standalone) and with this probe placed inside a catheter's lumen, to allow co-axiality with the catheter and possibly the catheter's visualization via the ultrasound as well.

In one aspect of the invention, a system for catheter placement may generally include a drilling device that may include a partial or full cannulation for placement of a device or object into the drilling device in a coaxial arrangement with the axis of a drill bit of the drilling device. In some embodiments, the cannulation may be present over at least a portion of the drilling device to accommodate insertion of a device or object, such as, for example, a navigation probe, wand or other navigation device for registering the position, orientation, trajectory or other spatial information of the drilling device. In some embodiments, the cannulation may run through the drilling device such that a device or object may be inserted into one end of the drilling device and may emerge from the other end, such as from an aperture at the end of a drill bit.

In another aspect of the invention, the system for catheter placement may further include a probe or other sensing device, such as a thin ultrasound probe or other thin sensing device, for imaging or otherwise detecting features of interest along the trajectory of the drilling device, such as blood vessels or other anatomical features. This may generally be desirable to aid in detecting and avoiding sensitive features (e.g. to avoid damaging blood vessels, nerves, other tissues, etc. along the trajectory). In some embodiments, the probe may be placed in the cannulation of the drilling device and not advanced out of the end to prevent damage during drilling. The probe may be advanced out of the end through the aperture, for example, after an access point has been drilled, and then utilized to detect and aid in guiding the trajectory of a placement procedure. In some embodiments, a catheter or needle may be inserted into the cannulation and the probe may be placed within the catheter or needle such that the probe is coaxial with the catheter or needle being placed in a tissue (e.g. for procedures at a target tissue such as aspiration, drainage, etc.).

In a further aspect of the invention, the probe may be utilized to visualize or detect features in the forward direction along the trajectory of the drilling device, circumferentially around the probe or a combination thereof. In some embodiments, an ultrasound probe may be utilized to detect blood vessels, nerves of interest, or other tissues of concern at its circumferential border as the probe and/or catheter/needle are advanced into a tissue (e.g. toward a brain ventricle, cyst, etc.). The detection or visualization of features of concern may then be utilized to alter the trajectory of the placement to avoid them, such as by backing out and altering the trajectory to avoid before advancing again.

In general, the drilling device, probe, needle and/or catheter may either be individually navigated for each component or as a unit or sub-combination.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

FIG. 1 illustrates an embodiment of the system of the present invention, with a full cannulation in a drilling device;

FIG. 1a illustrates a schematic view of the coaxial arrangement of a probe, deliverer (e.g. needle or catheter) and cannulation of a drilling device;

FIG. 1b illustrates simply the ultrasound probe inside the catheter (e.g. functioning as a stylet) after the hole has been drilled;

FIG. 2 illustrates an embodiment of the system of the present invention with a cannulated navigation device, a drilling guide and a catheter/needle/probe being placed into an anatomical structure;

FIG. 2a illustrates an alternative drilling guide in the form of a Thomale guide;

FIG. 2b illustrates a drilling device separate cannulation for a navigation device and for a catheter/needle/probe; and

FIGS. 3 and 3a illustrate a simulated ultrasound depiction of the (a) appearance of the item being delivered (needle or catheter or probe, etc.) on the screen, appearance of the blood vessel (to be avoided) with contrast enhancement, as depicted, or without and the ventricle or cyst or variable-echoic region (to be targeted).

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates to methods and devices for placement of catheter(s), more particularly to guided modality for catheter placement, and more particularly to guided modality placement of intracranial catheters with intra-luminal ultrasound, employing a drill, surgical navigation, or any combination thereof. In general, a device that may drill (with or without use of an external cranial inexpensive and portable guide, such as the Thomale Guide or other similar guide) may benefit from being cannulated, allowing placement of a thin or small imaging or sensing probe, such as, for example, a thin ultrasound probe, via a cannulated portion (or standalone) and with this probe placed inside a catheter's lumen, to allow co-axiality with the catheter.

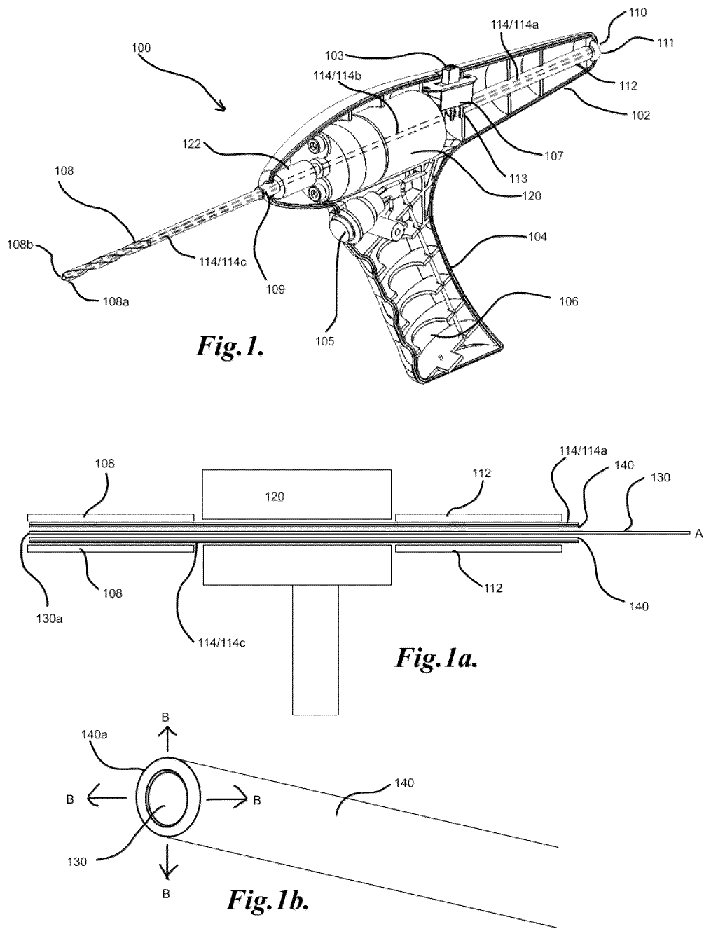

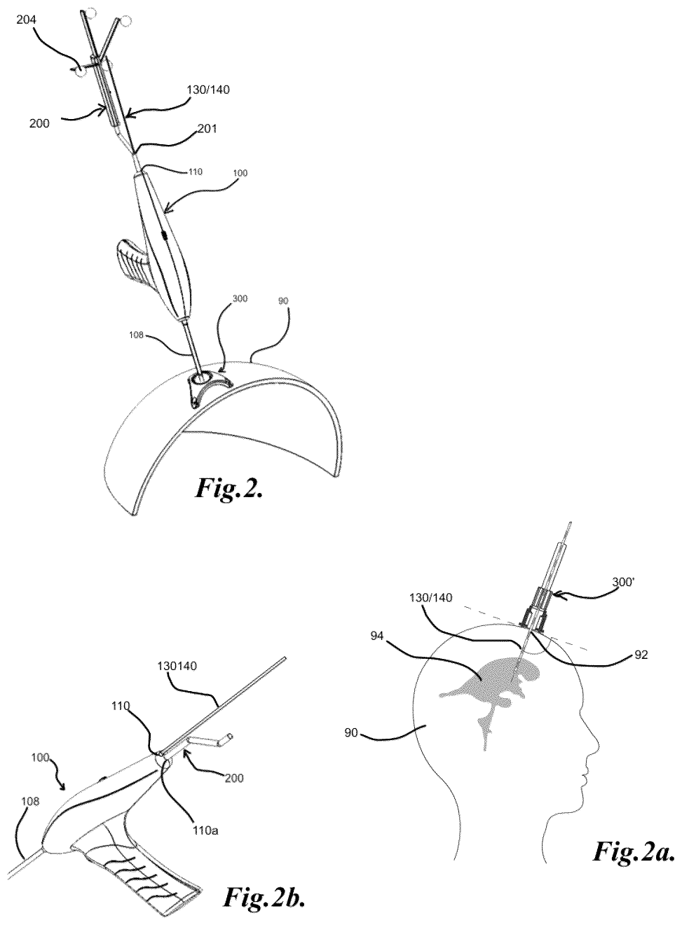

In one aspect of the invention, a system for catheter placement may generally include a drilling device that may include a partial or full cannulation for placement of a device or object into the drilling device in a coaxial arrangement with the axis of a drill bit of the drilling device. FIG. 1 illustrates a partial cut-away view of an embodiment of a drilling device 100 in the form of a surgical drill, which may generally include a main body 102 with a handle 104 for simple and precise use during a surgical procedure. The main body 102 and/or handle 104 may generally contain a motor 120, a power source (such as a battery which may be placed in space 106), a control system 107 for the motor 120 (as illustrated in connection with a direction switch 103). A drill bit and/or other tool, such as the drill bit 108 as illustrated in FIG. 1 with drilling features 108a, may be generally attached to the drill 100 through a mounting 109, such as reversibly by a chuck, or permanently by direct attachment, such as illustrated with attachment 122, to a gearing mechanism or to the motor 120, and may extend from one end of the main body 102 as illustrated. The drilling device 100 may further include an actuator, such as a trigger 105 or other appropriate control, for actuating the motor and the attached drill bit 108. The actuator may be present on the outside of the surgical drill 100, such as the trigger 105 is shown at the handle 104, or other portion of the main body 102, or other controls may be utilized, such as wired controls, wireless controls and/or other appropriate control mechanism. For example, foot pedals or other remote controls which may be common and familiar to surgeons may also be utilized. The drilling device 100 may also be employed with a drilling guide, such as the cranial guide 300 affixed to the skull 90 in FIG. 2, or the Thomale guide 300′ illustrated in FIG. 2a.

In some embodiments, a cannulation, shown as internal channel 114, may be present over at least a portion of the drilling device 100 to accommodate insertion of a device or object, such as, for example, a navigation probe, wand or other navigation device for registering the position, orientation, trajectory or other spatial information of the drilling device 100. FIG. 1 illustrates a cannulation as an internal channel 114 that runs through the drilling device 100 such that a device or object may be inserted into one end of the drilling device 100, such as at rear aperture 110 (illustrated with a retaining ring 111) through an interior conduit 112 (internal channel portion 114a), through a cannulation in the motor 120 (internal channel portion 114b) and may emerge from the other end, such as from an aperture 108b at the end of a drill bit 108 (internal channel portion 114c). In other embodiments, multiple cannulations may be provided in the drilling device 100, such as the cannulation 114 accessed through rear aperture 110 and a second cannulation accessed through aperture 110a as shown in FIG. 2b. The second cannulation may, for example, be used to accommodate a navigation device (e.g. wand 200 as illustrated), while the cannulation 114 accommodates a device (e.g. probe 130 and/or needle/catheter 140 as shown). In general, the second cannulation may not need to extend through the entire drilling device 100 to hold the navigation device 200 and any offset from the axis of the drill bit 108 may be predetermined for accurate navigation guidance.

In another aspect of the invention, the system for catheter placement may further include a probe or other sensing device, such as a thin ultrasound probe or other thin sensing device, for imaging or otherwise detecting features of interest along the trajectory of the drilling device, such as blood vessels or other anatomical features. This may generally be desirable to aid in detecting and avoiding sensitive features (e.g. to avoid damaging blood vessels, nerves, other tissues, etc. along the trajectory).

FIG. 1a shows a schematic view of the drilling device 100 with an inserted device, as illustrated with the catheter/needle 140 with a probe 130 within and coaxially with the internal channel 114. In some embodiments, the probe 130 may be placed in the cannulation 114 of the drilling device 100 and not advanced out of the end to prevent damage during drilling. The probe 130 may be advanced out of the end through the aperture, for example, after an access point has been drilled, and then utilized to detect and aid in guiding the trajectory of a placement procedure. In some embodiments, a catheter or needle 140 may be inserted into the cannulation 114 and the probe 130 may be placed within the catheter or needle 140 such that the probe 130 is coaxial with the catheter or needle 140 being placed in a tissue (e.g. for procedures at a target tissue such as aspiration, drainage, etc.). The probe 130 may further have a sensing element 130a that emerges in the tissue for detection and a connection at A to external equipment, such as a computer or other visualization device. In some embodiments, the probe 130 may utilize sensing technology that does not require it to emerge from the aperture 108b to function. FIG. 1b shows where, after drilling a hole, a probe 130, such as an ultrasound probe, placed within the catheter/needle 140 (e.g. functioning as a stylet, with borders 140a of the catheter 140 circumferentially visualized (shown with sensing indicators B) around the ultrasound image periphery as a possibility) would allow placement to target structure(s) while avoiding unintended traversal via structure(s) to be avoided.

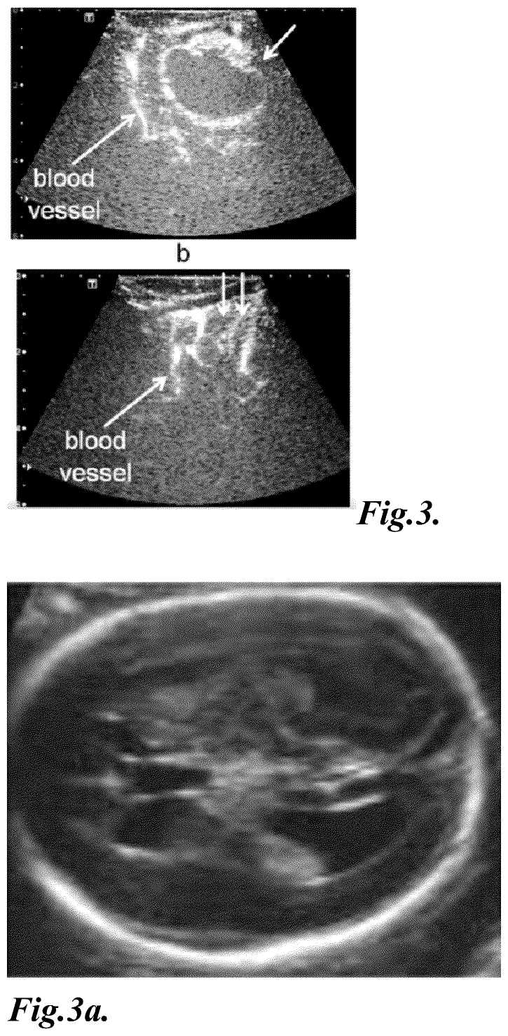

In a further aspect of the invention, the probe 130 may be utilized to visualize or detect features in the forward direction along the trajectory of the drilling device 100, circumferentially around the probe 130 or a combination thereof. In some embodiments, an ultrasound probe may be utilized to detect blood vessels, nerves of interest, or other tissues of concern at its circumferential border as the probe and/or catheter/needle are advanced into a tissue (e.g. toward a brain ventricle, cyst, etc.). The detection or visualization of features of concern may then be utilized to alter the trajectory of the placement to avoid them, such as by backing out and altering the trajectory to avoid before advancing again. Contrast agents may also be employed, as illustrated with the contrast enhanced ultrasound example in FIG. 3a.

In general, the drilling device 100, probe 130, needle and/or catheter 140 may either be individually navigated for each component or as a unit or subcombination. FIGS. 2 and 2b illustrate the use of navigation devices, such as wands 200 (which may also include fiducial markers 204 as illustrated). Navigation devices may also be placed on other parts of the drilling device 100 or on the catheter/needle 140 or probe 130. In some embodiments, the navigation device 200 may be placed in a cannulation, such as at rear aperture 110 and replaced with the probe 130/needle/catheter 140 during portions of the procedure. In some embodiments, a drilling device 100 may include multiple cannulations to accommodate both the navigation device 200 and the probe 130/needle/catheter 140 at the same time as illustrated in FIG. 2b and discussed above. In other embodiments, a cannulated navigation device 200 may be utilized, as illustrated with probe 130/needle/catheter 140 feeding into the cannulation 201 of navigation wand 200 in FIG. 2.

Example of Device and Method Use to Place a Ventricular Catheter into the Brain

As shown in FIGS. 2, 2a, probe 130, such as an ultrasound probe, placed inside a catheter 140 (or needle) is readied, with a hole 92 drilled in the skull 90 using a possibly cannulated and/or navigated/guided drill (e.g. drilling device 100 with aid of a cranial guide 300 or Thomale guide 300′), with goal of aiming a trajectory toward a target (in this case the ventricle 94 shown in FIG. 2a or on the example ultrasound image of FIG. 3a) with the bony hole 92 facilitating brain access in a path for the catheter 140 to follow towards the ventricle 94, while avoiding blood vessels.

Once the hole 92 is drilled (with either guided or non-guided drill), the soft tissue is traversed, with the ultrasound probe 130 placed into the delivering catheter 140 in such a manner that the catheter's wall is visualized around the periphery of the ultrasound image, while distally the ventricle 94 is seen deep, whereas any blood vessels are also seen (e.g. with hyperechoic wall and hypoechoic fluid), examples of which are illustrated in the ultrasound images of FIGS. 3 and 3a; the trajectory can be adjusted accordingly to allow an extrapolated pathway that avoids the vessels while reaching the target, by continuing along the chosen path—this method can be used to initially enter the brain parenchyma, then followed and backed-out if a vessel unexpectedly is encountered as further traversed, and path reset to avoid this vessel.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention. As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A method for stereotactic placement of a catheter into a body cavity comprising:
providing a probe inside a lumen of a catheter, said probe functioning as a stylet for said catheter;
providing a drilling device comprising:
a main body mounting a drill bit towards one end of the main body; and
a handle portion extending from said main body;
aligning the drill bit of said surgical drill along a predetermined trajectory into a body cavity using a navigation procedure;
drilling an access hole into said body cavity with said drill bit;
advancing said probe toward or into said body cavity from said aperture;
detecting the presence of any tissues of concern around said probe while advancing toward a target;
realigning of said predetermined trajectory to avoid any detected said tissues of concern near said probe; and
advancing of said catheter to said target via said realigning.

2. The method of claim 1, wherein said body cavity is a skull.

3. The method of claim 1, wherein said tissues of concern are selected from the group consisting of blood vessels and nerves.

4. The method of claim 1, wherein said probe comprises an ultrasound probe.

5. The method of claim 1, wherein said target comprises a brain ventricle or cyst.

6. The method of claim 1, wherein said drilling device further comprises a channel inside said main body and said drill bit, said channel extending through said main body and terminating at an aperture in said drill bit, said channel being coaxial with said drill bit.

7. The method of claim 6, wherein said probe and catheter are inserted into said channel to advance into said access hole.

8. A system for stereotactic placement of a catheter into a body cavity comprising:
a drilling device comprising:
a main body mounting a drill bit towards one end of the main body;
a handle portion disposed substantially toward said one end of the main body; and
a channel inside said main body and said drill bit, said channel extending through said main body and terminating at an aperture in said drill bit, said channel being coaxial with said drill bit;
a catheter placed in said channel and a probe placed in said catheter; and
a navigation system adapted to align said drilling device along a trajectory.

9. The system of claim 8, wherein said probe comprises an ultrasound probe.

10. The system of claim 8, wherein said navigation system comprises a frameless stereotactic navigation system comprising a navigation wand attached to said drilling device.

11. The system of claim 10, wherein said navigation wand comprises a cannulation that aligns with said channel and adapted to receive said probe and catheter.
